(19) United States
(12) Patent Application Publication
(10) Pub. No.: US 2023/0215531 A1
(43) Pub. Date: Jul. 6, 2023

(54) INTELLIGENT ASSESSMENT AND ANALYSIS OF MEDICAL PATIENTS

(71) Applicant: NuVasive, Inc., San Diego, CA (US)

(72) Inventors: Éric CHEVALIER, San Diego, CA (US); Naïm JALAL, San Diego, CA (US)

(21) Appl. No.: 18/000,751

(22) PCT Filed: Jun. 16, 2021
(86) PCT No.: PCT/IB2021/055290

§ 371 Date: Dec. 5, 2022

Related U.S. Application Data
(60) Provisional application No. 63/039,973, filed on Jun. 16, 2020.

(51) Int. Cl. G16H 15/00; G16H 50/20; G16H 50/70; G16H 30/20; G16H 40/67; G16H 10/60

(52) U.S. Cl. CPC G16H 15/00; G16H 50/20; G16H 50/70; G16H 30/20; G16H 40/67; G16H 10/60

(57) ABSTRACT

Systems and methods describe providing for the intelligent assessment and analysis of medical patient data. In one embodiment, the system receives medical imaging data of a patient, as well as connected implant data from an implant device implanted in the patient. A number of features are extracted via artificial intelligence (AI) algorithms from the medical imaging data and connected implant data. One or more reports are then generated based on the extracted features. In some embodiments, the systems and methods provide for indices, features, information, and/or metrics which have clinical value, and which enable a surgeon to support his or her decisions (related to, e.g., diagnosis, prognosis, monitoring, or any other suitable subject area).

### CROSS-REFERENCE TO RELATED APPLICATION

This application claims the benefit of U.S. Provisional Application No. 63/039,973, filed Jun. 16, 2020, which is hereby incorporated by reference in its entirety.

### FIELD

The presently described systems and methods relate generally to the medical field, and more particularly, to providing for intelligent assessment and analysis of medical patients.

### BACKGROUND

Within the medical field, artificial intelligence (AI) approaches and techniques have been significant and fruitful in solving a number of significant challenges. For example, deep learning, which is a form of machine learning (ML) wherein the parameters of the model are iteratively adjusted by the underlying algorithms based on the inputs and outputs to the model, has become the most used approach in medical image analysis.

The recent emergence of deep learning as a solution in medical image analysis is broadly due to the use of convolutional neural networks (CNNs), which allow for high performance in image processing problems. Within recent years, research has indicated that CNN approaches have matched the performance of experts while improving on their accuracy. Within dermatology, recent research showed that a single CNN trained on general skin classification was capable of achieving results on par with twenty-one dermatologists, tested across three critical diagnostic tasks. Furthermore, a CNN trained on a larger database exhibited better results than experts in terms of both specificity and sensitivity.

There are several drawbacks to the use of such AI models for medical diagnosis and assessment. First, a very large amount of data is required to train the model. A lack of sufficient data will lead to poorly trained models and consequently, poor diagnoses and assessments. Second, there is a lack of interpretability of these AI models. In a situation where one or more models disagree in suggestion or recommendation, there is no easy way to resolve the disagreement or explicate why a specific diagnosis was chosen. Third, there is poor generalization to new sets of data. It is crucial to carefully select training data sets to be the most representative of the task the algorithm will be trained to perform. An algorithm trained on pooled data from one site will often be less performant on another site, since there are differences in hospital methodologies (e.g., imaging or data collection processes), specific populations in hospitals, and more.

Thus, there is a need in the medical field to create a new and useful system and method for providing for assessment and analysis of medical patients. The source of the problem, as discovered by the inventors, is a lack of sufficiently large training data, a lack of interoperability between diagnostic models, and poor generalization to new sets of data. Key benefits of such a system and method include improved clinical outcomes for patients, increased knowledge of physiological processes, and significant improvements in medical practice via digital medicine.

### SUMMARY

The systems and methods described herein provide for intelligent assessment and analysis of medical patient data. In one embodiment, the system receives medical imaging data of a patient, as well as connected implant data from an implant device implanted in the patient. A number of features are extracted via artificial intelligence (AI) algorithms from the medical imaging data and connected implant data. One or more reports are then generated based on the extracted features. In some embodiments, the systems and methods provide for indices, features, information, and/or metrics which have clinical value, and which enable a surgeon to support his or her decisions (related to, e.g., diagnosis, prognosis, monitoring, or any other suitable subject area).

In some embodiments, matching similarities are determined by comparing the extracted features to a number of other features from previous patient data associated with one or more additional patients. The matching similarities are further used in generating the reports. In some embodiments, the systems additionally receives invasive data and extracts features from that data. In some embodiments, one or more of these steps are performed by one or more trained AI models.

Some embodiments relate to training AI models for performing one or more of the steps. In some embodiments, the trained is performed using one or more transfer learning datasets which are unrelated to the tasks the AI model is performing. In some embodiments, one or more training datasets are based on synthetic data related to one or more synthetic models.

Some embodiments relate to assessment and analysis of bone regeneration procedures. The extracted features may relate to bone regeneration, and the generated reports can include a number of bone regeneration metrics.

Some embodiments relate to optimizing distraction osteogenesis parameters. These embodiments may further include initializing a number of distraction osteogenesis parameters, predicting bone regeneration indices based on the distraction osteogenesis parameters, and generating optimized distraction osteogenesis parameters based on the predicted bone regeneration indices.

The features and components of these embodiments will be described in further detail in the description which follows. Additional features and advantages will also be set forth in the description which follows, and in part will be implicit from the description, or may be learned by the practice of the embodiments.

### BRIEF DESCRIPTION OF THE DRAWINGS

FIG. 1A is a diagram illustrating an exemplary environment in which some embodiments may operate.

FIG. 1B is a diagram illustrating an exemplary computer system that may execute instructions to perform some of the methods therein.

FIG. 2A is a flow chart illustrating an exemplary method that may be performed in accordance with some embodiments.

FIG. 2B is a flow chart illustrating additional steps that may be performed in accordance with some embodiments.

FIG. 2C is a flow chart illustrating additional steps that may be performed in accordance with some embodiments.

FIG. 3 is a flow chart illustrating an example embodiment of a method for providing assessment and analysis of a medical patient, in accordance with some aspects of the systems and methods herein.

FIG. 4 is a flow chart illustrating an example embodiment of a method for providing assessment and analysis of a medical patient, in accordance with some aspects of the systems and methods herein.

FIG. 5 is a flow chart illustrating an example embodiment of a method for providing assessment and analysis of a medical patient, in accordance with some aspects of the systems and methods herein.

FIG. 6 is a diagram illustrating an exemplary computer that may perform processing in some embodiments.

### DETAILED DESCRIPTION

In this specification, reference is made in detail to specific examples of the systems and methods. Some of the examples or their aspects are illustrated in the drawings.

For clarity in explanation, the systems and methods herein have been described with reference to specific examples, however it should be understood that the systems and methods herein are not limited to the described examples. On the contrary, the systems and methods described herein cover alternatives, modifications, and equivalents as may be included within their respective scopes as defined by any patent claims. The following examples of the systems and methods are set forth without any loss of generality to, and without imposing limitations on, the claimed systems and methods. In the following description, specific details are set forth in order to provide a thorough understanding of the systems and methods. The systems and methods may be practiced without some or all of these specific details. In addition, well known features may not have been described in detail to avoid unnecessarily obscuring the systems and methods.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

The following generally relates to the intelligent assessment and analysis of medical patients.

I. Example Use Case

One example use case for the systems and methods herein relates to the need for evaluation and monitoring of bone regeneration in a medical patient using artificial intelligence models. This may involve bone regeneration areas and procedures such as, e.g., spinal fusion, distraction osteogenesis, fracture healing, and more. Within spinal fusion, for example, there is a high rate of non-union. One current gold standard practice is exploratory surgery, which is invasive, costly, and often unethical to perform on patients not exhibiting symptoms.

Another current practice is for a clinician to acquire patient data to support making a diagnosis. For example, a clinician may acquire medical images such as a CT scan, and use the CT scan with an irradiating device. The clinician may then combine this data with non-invasive patient data, such as biometric or clinical data. Based on this, the clinician makes a diagnosis (e.g., full weight bearing) and then writes a report including the diagnosis. This current practice leads to misdiagnoses much of the time. Within the context of distraction osteogenesis, for example, there is no solution for supporting a decision for distraction, such as full weight bearing. Rather, data such as x-rays and CTs have proven to be inexact and unreliable. For fracture healing, the current practice of physical examinations and medical imaging leads to high complication rates. X-rays and CT scans have low reliability, and ultrasonography relies heavily on the skills of a sonologist.

With the systems and methods herein, however, an additional type of data, connected implant data, is acquired. Connected implant data is generated by an implantable device with at least one sensor, which can communicate with an external device and provide information to, e.g., a clinician or caregiver. Features of interest are extracted via AI algorithms based on these pieces of data, including the connected implant data. In some embodiments, these features are then compared to previous features, e.g., from a feature repository of previous cases, and matching similarities are determined. A report is then generated, including, e.g., an assessment (e.g., diagnosis) of the medical patient with respect to his or her condition and/or pathology.

II. Definitions

“Artificial intelligence” (AI) methods, processes, techniques, models, or algorithms may refer variously to symbolic processes, numerical processes, or a combination thereof. Symbolic AI methods may include, e.g., system expert, decision tree, fuzzy logic, rule-based systems, or any other suitable symbolic AI methods. Numerical AI methods may refer to any form of supervised or unsupervised learning, including, e.g., logistic regression, support vector machines, K-means clustering, evolutionary methods, convolutional neural networks (CNNs), recurrent neural networks (RNNs), any other suitable form of neural network, or any other suitable numerical AI methods.

“Medical imaging data” refers to any images of the human anatomy obtained through a medical imaging modality for the purpose of diagnosis, prognosis, monitoring. This data may relate to static or dynamic x-ray images, computerized tomography (CT) scans, single-photon emission computerized tomography (SPECT) scans, scintigraphy, magnetic resonance imaging (MRI) images.

“Non-invasive patient data” refers to implantable wearable sensor data, biometric data, and/or non-invasive medical examination data (e.g., relating to propaedeutic procedures, electrographs, or any other non-invasive medical examination data). Additionally, non-invasive patient data can include information relating to a patient's past and/or current health or illness, their treatment history, lifestyle choices, or other history information.

“Invasive patient data” refers to previously obtained data, per-surgery data and/or post-surgery data gathered through a medical procedure that requires a cut skin on the examined patient. This data may relate to, e.g., biological state, and/or inherited or acquired genetic characteristics. In some embodiments, invasive patient data may include, e.g., bone tissue biomarkers or genetic data blood tests.

“Connected implant data” refers to patient data relating to or originating from a connected implant which is implanted in the patient. In some embodiments, connected implant data may include data on the location, etiology, and severity of pathology, the indication, or the connected implant environment, or any other patient-specific connected implant data.

“Wearable sensor” refers to sensors integrated into wearable objects or integrated directly with the body, from which patient data can be obtained which relates to, e.g., the sensor itself, the activity, the behavior or the treatment follow-up, or any other suitable patient data or information.

“Bone regeneration” refers to a physiological process of bone formation occurring, for instance, during spinal fusion, fracture healing, or distraction osteogenesis.

“Bone bridging area” refers to the bone area at a given level providing a mechanical link between the adjacent vertebrae or between bone ends. Bone bridging area could be only one area or the sum of several areas providing the mechanical link.

Other definitions and terms are discussed and provided within the present specification based on context.

III. Exemplary Environments

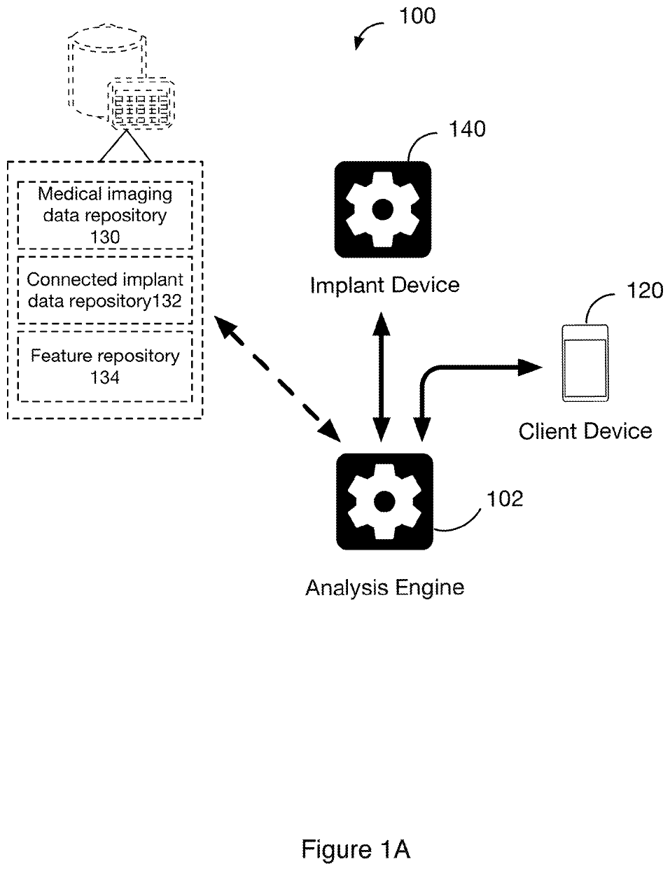

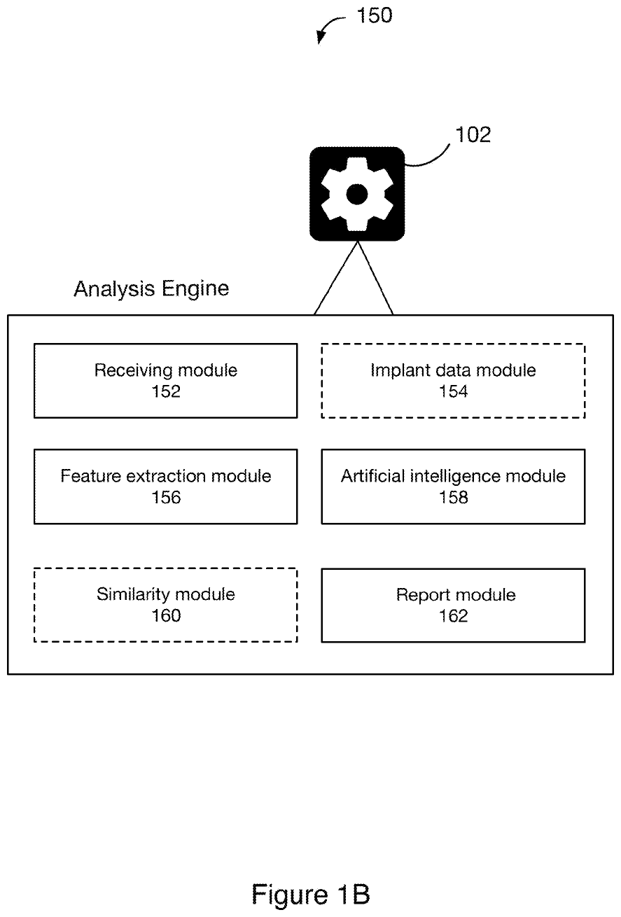

FIG. 1A is a diagram illustrating an exemplary environment in which some embodiments may operate. In the exemplary environment 100, a client device 120 is connected to an analysis engine 102. The analysis engine 102 is optionally connected to one or more optional database(s), including a medical imaging data repository 130, connected implant data repository 132, and/or feature repository 134. One or more of the databases may be combined or split into multiple databases. The analysis engine 102 is connected to an implant device 140. The implant device 140 and/or client device 120 in this environment may be computers.

The exemplary environment 100 is illustrated with only one client device and analysis engine for simplicity, though in practice there may be more or fewer client devices and/or analysis engines. In some embodiments, the client device and analysis engine may be part of the same computer or device.

In an embodiment, the analysis engine 102 may perform the method 200 or other method herein and, as a result, provide assessment and analysis of medical patients. In some embodiments, this may be accomplished via communication with the client device, implant device 140, and/or other device(s) over a network between the client device 120, implant device 140, and/or other device(s) and an application server or some other network server. In some embodiments, the analysis engine 102 is an application hosted on a computer or similar device, or is itself a computer or similar device configured to host an application to perform some of the methods and embodiments herein.

Client device 120 is a device that sends and receives information to the analysis engine 102. In some embodiments, client device 120 is a computing device capable of hosting and executing one or more applications or other programs capable of sending and receiving information. In some embodiments, the client device 120 may be a computer desktop or laptop, mobile phone, virtual reality or augmented reality device, wearable, or any other suitable device capable of sending and receiving information. In some embodiments, the analysis engine 102 may be hosted in whole or in part as an application executed on the client device 120.

Implant device 140 refers to a connected implant, i.e., an implantable device implanted in a patient. In some embodiments, the implant device 140 includes at least one sensor for generating and/or obtaining connected implant data. In some embodiments, the implant device 140 is configured with the ability to communicate the connected implant data to one or more devices or computers which are external to the patient.

In various embodiments, the sensor(s) in the implant device 140 may be one or more of, e.g., a force sensor, strain gauge, piezoelectric accelerometer, temperature sensor, potential hydrogen (pH) sensor, ultrasonic sensor, ultra-wideband radar, hall effect sensor, capacitive displacement sensor, oxygen sensor, biosensor, or any other suitable sensor, radar, or transducer.

In some embodiments, the connected implant data may be raw signals in the frequency or time domain (i.e., 1 to nD frame of figures). Additionally or alternatively, connected implant data may be precomputed values or signals at one or more locations, such as, e.g., force, stress, elastic modulus, displacement, pH, or any other suitable biological, physical, and/or chemical observable values or signals which could be associated with or related to a medical procedure performed on the patient.

Optional database(s) including one or more of a medical imaging data repository 130, connected implant data repository 132, and/or feature repository 134. These optional databases function to store and/or maintain, respectively, medical imaging data, connected implant data, and features of interest extracted from one or more pieces of patient data. In some embodiments, non-invasive patient data may be stored in a non-invasive patient data repository. In another embodiment, invasive patient data may be stored in a invasive patient data repository. The optional database(s) may also store and/or maintain any other suitable information for the analysis engine 102 to perform elements of the methods and systems herein. In some embodiments, the optional database(s) can be queried by one or more components of system 100 (e.g., by the analysis engine 102), and specific stored data in the database(s) can be retrieved.

FIG. 1B is a diagram illustrating an exemplary computer system that may execute instructions to perform some of the methods therein. The diagram shows an example of an analysis engine configured to assess and analyze a medical patient and generate one or more reports based on the assessment and analysis. Analysis engine 150 may be an example of, or include aspects of, the corresponding element or elements described with reference to FIG. 1A. In some embodiments, analysis engine 150 is a component or system on an enterprise server. In other embodiments, analysis engine 150 may be a component or system on client device 120, or may be a component or system on peripherals or third-party devices. Analysis engine 150 may comprise hardware or software or both.

In the example embodiment, analysis engine 150 includes receiving module 152, optional implant data module 154, feature extraction module 156, artificial intelligence module 158, optional similarity module 160, and report module 162.

Receiving module 152 functions to receive data from other devices and/or computing systems via a network. The data received includes patient data relating to a patient. In various embodiments, the patient data may include medical imaging data, invasive patient data, non-invasive patient data, connected implant data, or any other suitable form of patient data. In some embodiments, the network may enable transmitting and receiving data from the Internet. Data received by the network may be used by the other modules. The modules may transmit data through the network.

Optional implant data module 154 functions to process connected implant data received by the receiving module 152. In some embodiments, the connected implant data is generated by one or more sensors embedded within the connected implant device. In some embodiments, the implant data module 154 processes the connected implant data by receiving the data from the implant device, classifying the data into one of multiple predefined categories for connected implant data, converting the data into a format appropriate for that category of data, and storing the data in an appropriate database.

In some embodiments, the implant data module 154 normalizes the connected implant data in one or more ways. In some embodiments, the implant data module 154 prunes any unnecessary data from the received connected implant data. In some embodiments, receiving module 152 and/or implant data module 154 may remove and/or modify Personal Identifiable Information (PII) from data in an anonymization or pseudonymization step. Normalized connected implant data may then be passed to the feature extraction module 156 and/or artificial intelligence module 158 for further processing.

Feature extraction module 156 functions to extract one or more features of interest of the received data which was received by receiving module 152, which will be described in further detail below.

Artificial intelligence module 158 functions to perform artificial intelligence tasks. In various embodiments, such tasks may include various machine learning, deep learning, and/or symbolic artificial intelligence tasks within the system. In some embodiments, artificial intelligence module may include training one or more artificial intelligence models. The artificial intelligence module 158 may comprise decision trees such as, e.g., classification trees, regression trees, boosted trees, bootstrap aggregated decision trees, random forests, or a combination thereof. Additionally or alternatively, artificial intelligence module 158 may comprise neural networks (NN) such as, artificial neural networks (ANN), autoencoders, probabilistic neural networks (PNN), time delay neural networks (TDNN), convolutional neural networks (CNN), deep stacking networks (DSN), radial basis function networks (RBFN), general regression neural networks (GRNN), deep belief networks (DBN), deep neural networks (DNN), deep reinforcement learning (DRL), recurrent neural networks (RNN), fully recurrent neural networks (FRNN), Hopfield networks, Boltzmann machines, deep Boltzmann machines, self-organizing maps (SOM), learning vector quantizations (LVQ), simple recurrent networks (SRN), reservoir computing, echo state networks (ESN), long short-term memory networks (LSTM), bi-directional RNNs, hierarchical RNNs, stochastic neural networks, genetic scale models, committee of machines (CoM), associative neural networks (ASNN), instantaneously trained neural networks (ITNN), spiking neural networks (SNN), regulatory feedback networks, neocognitron networks, compound hierarchical-deep models, deep predictive coding networks (DPCN), multilayer kernel machines (MKM), cascade correlation networks (CCN), neuro-fuzzy networks, compositional pattern-producing networks, one-shot associative memory models, hierarchical temporal memory (HTM) models, holographic associative memory (HAM), neural Turing machines, or any combination thereof. In some embodiments, mathematical tools may also be utilized in performing artificial intelligence tasks, including metaheuristic processes such as, e.g., genetic processes, great deluge processes, and/or statistical tests such as Welch's t-tests or F-ratio tests. Any other suitable neural networks, mathematical tools, or artificial intelligence techniques may be contemplated.

A neural network is a hardware or a software component that includes a number of connected nodes (a.k.a., artificial neurons), which may be seen as loosely corresponding to the neurons in a human brain. Each connection, or edge, may transmit a signal from one node to another (like the physical synapses in a brain). When a node receives a signal, it can process the signal and then transmit the processed signal to other connected nodes. In some embodiments, the signals between nodes comprise real numbers, and the output of each node may be computed by a function of the sum of its inputs. Each node and edge may be associated with one or more node weights that determine how the signal is processed and transmitted.

In some embodiments, during the training process for an artificial intelligence model, the artificial intelligence module 156 may adjust these weights to improve the accuracy of the result (e.g., by minimizing a loss function which corresponds in some way to the difference between the current result and the target result). The weight of an edge may increase or decrease the strength of the signal transmitted between nodes. In some embodiments, nodes may have a threshold below which a signal is not transmitted at all. The nodes may also be aggregated into layers. Different layers may perform different transformations on their inputs. In some embodiments, the initial layer is the input layer and the last layer is the output layer. In some cases, signals may traverse certain layers multiple times.

In some embodiments, training the artificial intelligence models is performed using one or more datasets based on synthetic data, where the synthetic data is related to one or more synthetic models. The training can include generating patient-specific synthetic geometrics based on features extracted from the medical imaging data, then generating one or more synthetic models based on the synthetic geometries and the indices. One or more measures are extracted from the one or more synthetic models. In some embodiments, the measures are comparable (e.g., similar or identical) to those used to measure features of interest using the connected implant. Finally, the algorithm is trained to output indices from the synthetic geometries and the measures. In some embodiments, the indices are bone regeneration indices.

Optional similarity module 160 functions to compare the extracted features to a number of other features from previous patient data associated with one or more additional patients in order to determine a plurality of matching similarities, which will be described in further detail below.

Report module 162 functions to generate one or more reports based on the extracted features from feature extraction module 156, and/or optionally the matching similarities of optional similarity module 160 and/or output from artificial intelligence model 158. This report generation will be described in further detail below.

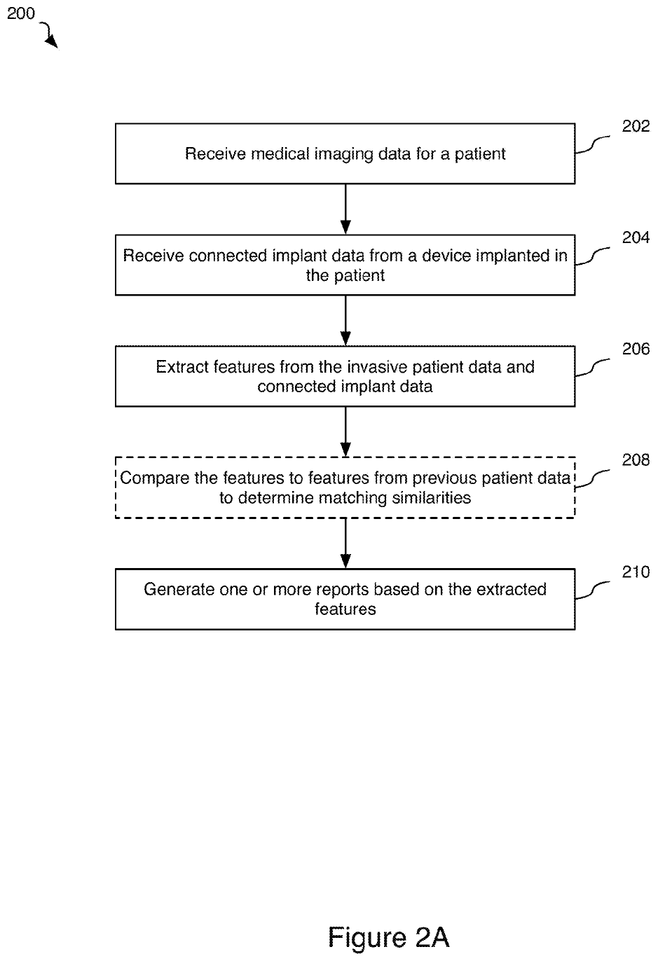

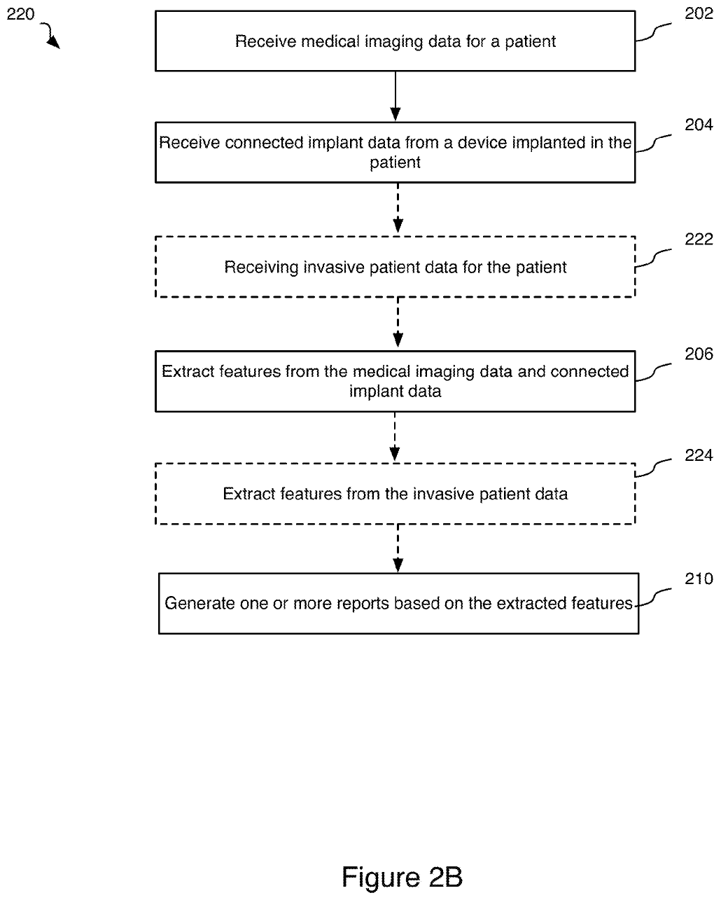

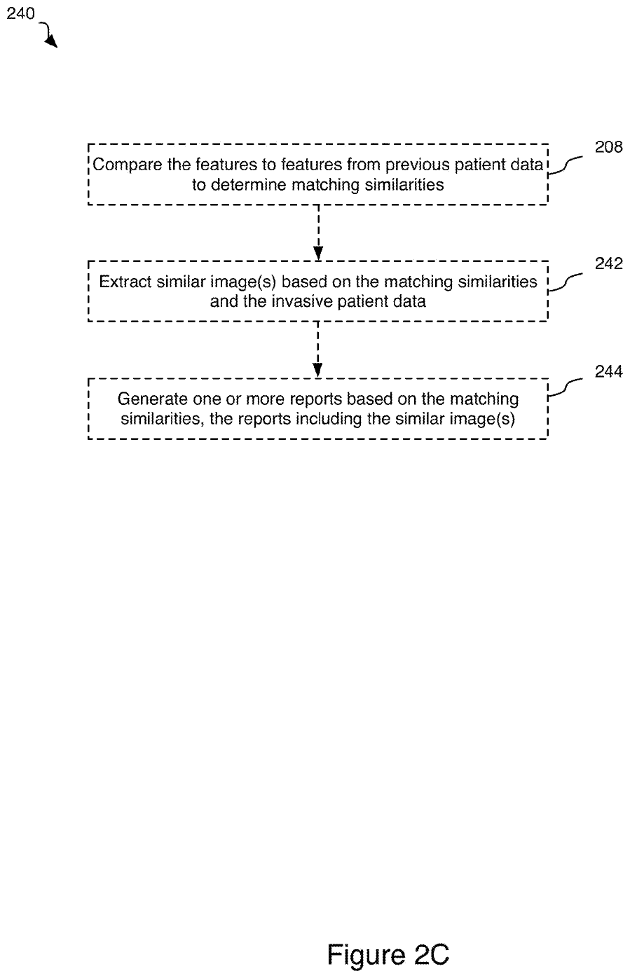

Within the following FIGS. 2A, 2B, and 2C, the order of the steps illustrated can be contemplated to be different. For example, in one embodiment of FIG. 2A, steps 202 and 204 are performed concurrently in parallel, then step 206, step 208, and step 210 are performed sequentially.

FIG. 2A is a flow chart illustrating an exemplary method that may be performed in accordance with some embodiments. The flow chart shows an example of a process for providing assessment and analysis of a patient. In some examples, these operations may be performed by a system including a processor executing a set of codes to control functional elements of an apparatus. Additionally or alternatively, the processes may be performed using special-purpose hardware. Generally, these operations may be performed in accordance with some aspects of the systems and methods herein. For example, the operations may be composed of various substeps, or may be performed in conjunction with other operations described herein.

At step 202, the system receives medical imaging data for a patient. This data includes one or more medical images of the patient. Medical imaging data can be previously obtained imaging data, per-surgery imaging data, or post-surgery imaging data. In some embodiments, such as those which relate to bone regeneration, medical imaging data includes bone tissue biomarkers. In some embodiments, the system receives the medical imaging data from a client device, analysis engine, database, or other device, computer, engine, or repository.

In some embodiments, the system additionally or alternatively receives invasive patient data for the patient. Invasive patient data may include, e.g., previously obtained data, per-surgery data and/or post-surgery data gathered through a medical procedure that requires a cut skin on the examined patient. This data may relate to, e.g., biological state, and/or inherited or acquired genetic characteristics. In some embodiments, invasive patient data may include, e.g., bone tissue biomarkers or genetic data blood tests, or any other suitable invasive patient data.

In some embodiments, the system additionally or alternatively receives non-invasive patient data for the patient. Non-invasive patient data may include, e.g., patient conditions, biometric data, clinical examination data, wearable device data, or any other suitable non-invasive patient data.

At step 204, the system receives connected implant data for the patient from an implant device which is implanted in the patient. In some embodiments, connected implant data may be, patient data relating to or originating from the connected implant itself. In some embodiments, connected implant data may include data on the location, etiology, and severity of pathology, the indication, or the connected implant environment, or any other patient-specific connected implant data.

In varying embodiments, connected implant data may be precomputed data such as, e.g.: a single value (i.e., a temperature); a vector of figures in the time domain (i.e., the evolution of the elastic modulus at one particular point during a certain time period); a matrix of figures in the time domain (i.e., the evolution of the elastic modulus at one particular line during a certain time period); a three-dimensional (3D) frame of figures in the time domain (i.e., the evolution of the elastic modulus at one particular plane during a certain time period); a four-dimensional (4D) frame of figures in the time domain (i.e., the evolution of the elastic modulus at one particular volume during a certain time period); a five-dimensional (5D) frame of figures in the time domain (i.e., the evolution of several parameters at one particular volume during a certain time period); or any other suitable precomputed data.

At step 206, the system extracts features from at least the medical imaging data and connected implant data. Features refer to relevant characteristics, parameters, or criteria which factor into assessment and/or analysis of medical procedures. In some embodiments, the features are predicted, wherein the output prediction is from a machine learning model or other artificial intelligence model trained on a set of data (e.g., via artificial intelligence module 158). In some embodiments, determining and/or predicting the features can involve feature extraction processes and/or classification techniques employed by machine learning, computer vision, or other artificial intelligence processes or models. In some embodiments, the techniques can additionally or alternatively include object detection, object tracking, segmentation, and other known feature extraction techniques. In some embodiments, for received data constituting an image (e.g., an x-ray or other image data relating to a medical procedure), image detection and image analysis techniques may be employed to extract features.

In some embodiments, features may be extracted from invasive patient data, such as bone tissue biomarkers (BTMs) or genetic data. RNNs, symbolic processes, or some combination thereof could potentially be used for such applications. In some embodiments, patient conditions acquired in previous steps could be set as input into one or more AI models to address such problems as, e.g., external factors impacting bone tissue biomarker secretions. In some embodiments, regression or other techniques can be applied in order to extract features of interest.

In some embodiments, the system can additionally or alternatively extract features from non-invasive patient data in a similar fashion. For example, for wearable device data, AI models such as CNNs and RNNs may accept such inputs as inertial gait time-series signals or microelectromechanical sensory signals. Non-invasive features of interest, such as activity recognition and quantification, could be outputted from this set of AI models. In some embodiments, the features are extracted into a features vector constituting scalar values.

At optional step 208, in some embodiments, the system compares the extracted features to features from previous patient data in order to determine matching similarities. In some embodiments, the system compares a features vector obtained at step 206 with features and/or a features vector from a feature repository 134 or other database. In various embodiments, one or several process can be used to determine the similarity between feature sets or features vectors. In some embodiments, a mathematical tool including meta-heuristic processes, such as, e.g., genetic processes, great deluge processes, or statistical tests such as Welch's t-tests or F-ratio could be used. Additionally or alternatively, in some embodiments, structure element correlation, global correlation vector, or directional global correlation vector could be used separately or in combination.

In some embodiments, the determined matching similarities are ranked, scored, or otherwise assigned a numerical or qualitative value, such that some matching similarities are designating as, e.g., ranking or scoring higher than others depending on the extent of the determined similarity.

At step 210, the system generates one or more reports based on the extracted features. The report may be in any potential form and include various information. For example, in some embodiments, the report may include one or more images, three-dimensional reconstructions, tables, graphs, or other visual renderings or representations highlighting or displaying information with respect to identified, classified, or segmented targets. For example, in the context of bone regeneration procedures, proposed diagnostics or bone regeneration indices could be highlighted in a superpixel-based approach and/or heat map visualizations in order to direct the specialist's attention to the target. Thus, in the case of a pseudarthrosis diagnosis, for example, non-fusion zones could be overlaid on top of medical images.

FIG. 2B is a flow chart illustrating additional steps that may be performed in accordance with some embodiments. The flow chart shows an example of a process for providing assessment and analysis of a medical patient. Steps 202, 204, 206, and 210 are identical to the steps in FIG. 2A. Optional steps 222 and 224 have been added. At optional step 222, the system receives, in addition to medical imaging data at step 202 and connected implant data at step 204, invasive patient data for the patient. At optional step 224, in addition to extracting features from the medical imaging data and connected implant data at step 206, the system extracts features from the invasive patient data.

FIG. 2C is a flow chart illustrating additional steps that may be performed in accordance with some embodiments. The flow chart shows an example of a process for providing assessment and analysis of medical patient data. Optional steps 242 and 244 have been added.

At optional step 242, the system extracts similar image(s) based on the extracted features and the invasive patient data. In some embodiments, the similar images are images from one or more similar cases pertaining to previous patient data. In some embodiments, if the matching similarity is high between the extracted features of the patient data and the extracted features of a previous case, then the system extracts images from that previous case which may highlight or emphasize the similarities between the two feature sets. In some embodiments, the extraction process is performed offline. In some embodiments, in a later online process, a new image is received and features of interest are extracted from the image using the same process used in the offline process. This allows for the extraction of similar images, and allows caregivers and providers to support similar images for their diagnosis.

At optional step 244, the system generates one or more reports based on the extracted features, as in step 210 of FIG. 2A. The system additionally includes the similar image(s) from optional step 242 in the generated reports. In some embodiments, the desired number of similar images which are included in the report is optionally adjustable by one or more parties (e.g., the caregiver for the patient). In some embodiments, similar image(s) are sorted based on the relevance or similarity ranking of their associated cases.

In various embodiments, the generated report can include one or more of a medical diagnosis, prediction, identification of pathologies, conditions, or characteristics in one or more images, probability, expected timeline for recovery, or any other suitable information relevant to a report with respect to a medical patient. For example, a generated report may include a prediction of an appropriate time to remove a connected implant, such as osteosynthesis hardware (e.g., an osteosynthesis plate). The report may further include a suggestion of adapting degree of freedom for that particular osteosynthesis hardware during the fracture healing process. For this application, the report may indicate the probability of being within a different fracture healing stage. The report may also include a list of relevant features which explain the similarity between the current patient data and previous patient data. The report may additionally suggest a corrective action, e.g., bone grafting or adjustment of the degree of freedom of the osteosynthesis hardware.

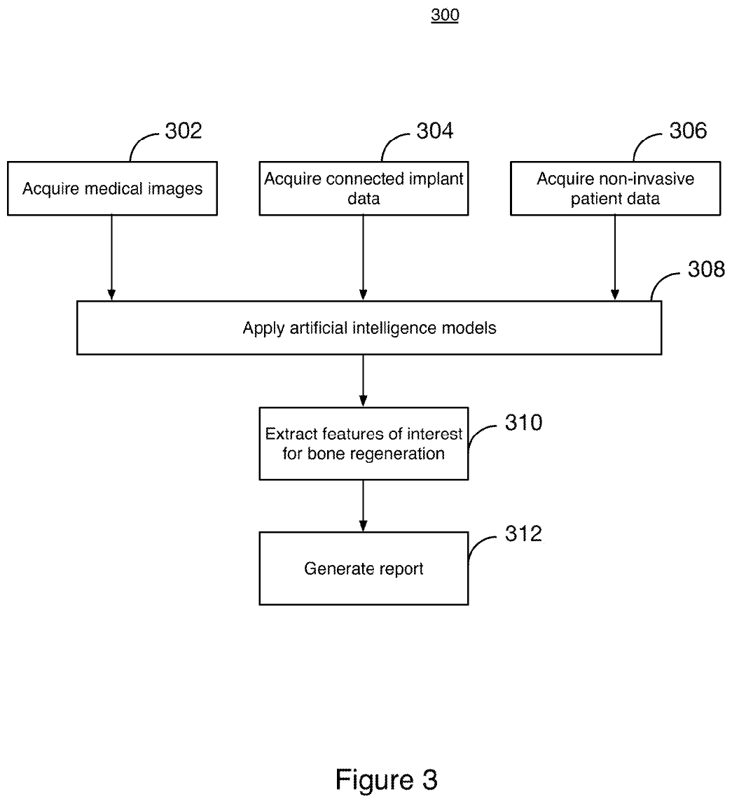

FIG. 3 is a flow chart illustrating an example embodiment of a method for providing assessment and analysis of medical patient data, in accordance with some aspects of the systems and methods herein. The example embodiment relates to bone regeneration. Specifically, the example embodiment includes data acquisition in steps 302, 304, and 306, application of one or more artificial intelligence models in step 308, classification of bone regeneration features of interest in step 310, and report generation in step 312.

At step 302, medical images are acquired. The medical images could be, e.g., computed tomography (CT), x-ray images (for example, static/flexion/extension, with or without contrast agents), magnetic resonance imaging (MRI) images, ultrasound or invasive imaging such as scintigraphy, single-photon emission CT (SPECT/CT) X-ray angiography, intravascular ultrasound (IVUS), optical coherence tomography (OCT), near-infrared spectroscopy and imaging (NIRS), or other types of medical images. In some embodiments, the image data consists of scalar values organized as a frame of data. Alternatively, image data could consist of raw data.

At step 304, connected implant data is acquired. The connected implant data could be, e.g., raw signals in the frequency or time domain. Alternatively, connected implant data could be precomputed values such as force, stress, elastic modulus, displacement or other values at one or more locations.

At step 306, non-invasive patient data is acquired. The non-invasive patient data can include, e.g., biometric data, which refers to any measurable physical characteristic that can be checked by a machine or computer. Additionally, it may include information relating to the patient's past and/or current health or illness, treatment history, lifestyle choices, or other history information. It may also include wearable sensor data or non-invasive medical examination data relating to, e.g., propaedeutic procedures, electrographs or other non-invasive medical examinations.

At step 308, one or more trained Artificial Intelligence models are applied on input data, wherein the input data consists of the acquired medical images, connected implant data and non-invasive patient data. Artificial Intelligence models could be symbolic processes or techniques such as, e.g., expert system or fuzzy logic, unsupervised machine learning models, supervised machine learning models such as logistic regression, support vector machines, neural networks including, for example, convolutional neural networks or recurrent neural networks, or other artificial intelligence models. In some embodiments, one or more numerical processes (e.g., machine learning models) are combined with symbolic processes such as expert system or fuzzy logic in order to profit from both the performance of numerical processes and the reasoning capabilities of symbolic processes. This hybrid approach could allow for an increase in output interpretability, which would thus address the typical lack of explicability in the previous state of the art.

At step 310, one or more features of interest relative to bone regeneration are extracted. In some embodiments, the application of at least one artificial intelligence model in step 308 can provide, e.g., bone regeneration analyses or bone regeneration indices. These may serve the function of supporting a caregiver diagnosis, prognosis, or treatment choice. For example, the trained artificial intelligence models may be designed to identify the presence of non-fusion zones based on only medical images or based on medical images, connected implant data, and non-invasive patient data. In another example, 3D mapping callus mechanical properties could be obtained at the output of the trained artificial intelligence models.

In step 312, a report may be generated based on the features of interest computed in step 310. The report in this example may include the features of interest as well as bone regeneration indices determined at step 310.

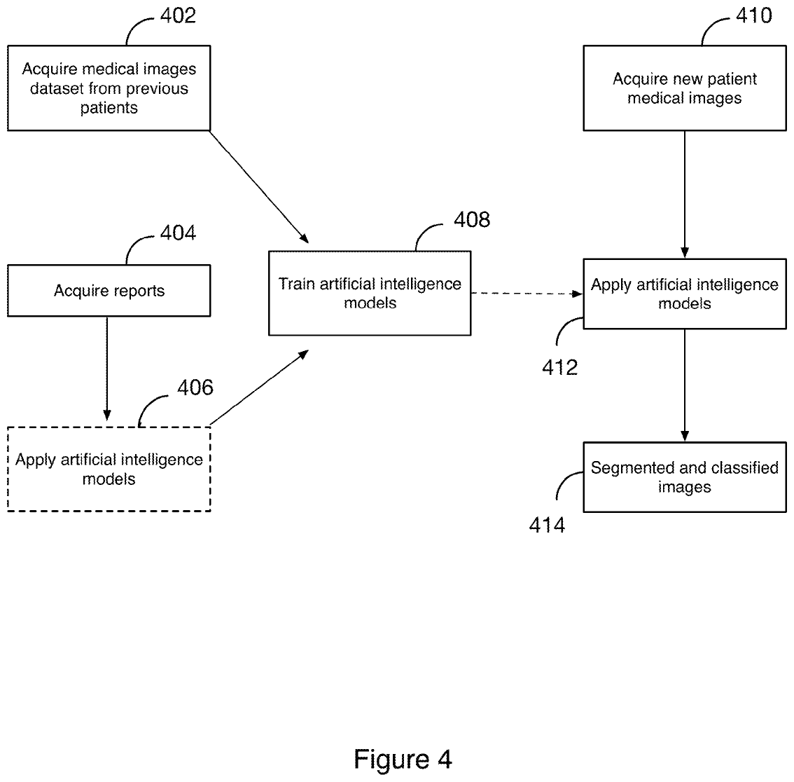

FIG. 4 is a flow chart illustrating an example embodiment of a method for providing assessment and analysis of a medical patient, in accordance with some aspects of the systems and methods herein. The example embodiment relates to training one or more artificial intelligence models to perform tasks pursuant to the systems and methods here. Specifically, steps 402 and 404, optional step 406, and step 408 constitute a training phase, while steps 410, 412, and 414 constitute a prediction phase (i.e., assessment and analysis performed by the trained artificial intelligence model or models).

At steps 402 and 404, medical images and imaging reports archived from previous patients concerning the specific target problem (e.g., a pathology or condition) are acquired from different hospitals or providers. In some embodiments, medical images are obtained using CT or other non-invasive and/or invasive imaging modalities. In some embodiments, the image data consists of scalar values organized as a frame of data. Additionally or alternatively, the image data can be in the raw data domain. Imaging reports thus inform clinical decision-making regarding different therapeutic approaches and are used to assess treatment responses. Alternatively, imaging reports can be annotated image(s) indicating, e.g., different tissues and target pathology areas. Alternatively, imaging reports can be structured data, e.g., a frame of figures, booleans, grades, and/or coordinates of the target pathology areas.

At optional step 406, one or more artificial intelligence (AI) models are applied to the imaging report to automatically extract a diagnosis. In some embodiments, location, etiology, and severity of pathology could be the output of the model. In some embodiments, one or more AI models may apply natural language processing. In some embodiments, recurrent neural networks (RNNs) such as long short-term memory processes (LSTM) or other AI models can be used to extract the target information for each imaging study. Alternatively to performing step 406, one or more pieces of received data can be designed as the target diagnosis (i.e., ground truth) for the training phase.

At step 408, one or more AI models are trained to output the target diagnosis, pathology areas, prognosis or any other suitable subject area (also referred as ground truth) from the medical images input. In some embodiments, the models can be convolutional neural networks (CNNs) or other AI techniques. In some embodiments, an end-to-end AI model can be trained with only one deep neural network. In some embodiments, tasks to be performed by the AI models can be subdivided into two or more tasks, such as, e.g., image enhancement, segmentation, and classification.

At step 410, medical images concerning the specific target problem are acquired from a new patient. At step 412, the AI models, which were trained at steps 402 and 404, optional step 406, and step 408, are applied to the new patient medical images. At step 414, the AI models output a diagnostic (and/or prognostic, pathology area, or any other suitable subject area) assessment report containing the segmented and classified images.

In some embodiments, one or more additional steps for transfer learning are performed in relation to the training steps. Transfer learning is a technique developed to address the need for a large amount of training data in order to sufficiently train an AI model. Transfer learning involves initially pre-training the AI model (e.g., a deep neural network) with a huge dataset that is unrelated to the task of interest, and then fine-tuning only the deeper layer parameters with the data from the task of interest. In some embodiments, each of one or more transfer learning methods can include its own transfer learning dataset. In other words, the “transfer learning” is the method or task allowing the AI model to pre-learn, and it uses a dataset which can often be subsequently different from the actual dataset of the application.

In some embodiments, a large labeled dataset of images is acquired. The dataset may be acquired from, e.g., an open database, such as ImageNet. In some embodiments, images are not necessarily medical images, but in other embodiments, images could be exclusively or not exclusively medical images. A large dataset could potentially amount to several million images, or alternatively a dataset could amount to fewer or larger image quantities.

In some embodiments, there are multiple layers of learning occurring during the training process. For example, two layers can involve transfer learning, with a third providing final learning. In some embodiments, transfer learning includes acquiring very large datasets or images, acquiring labels for the images, and training an AI model based on these labeled datasets. Some of the parameters initialized during the training for this AI model may then be used as initialization parameters for the next AI model set to be trained with higher optimization. This process can continue for training a number of AI models within the system.

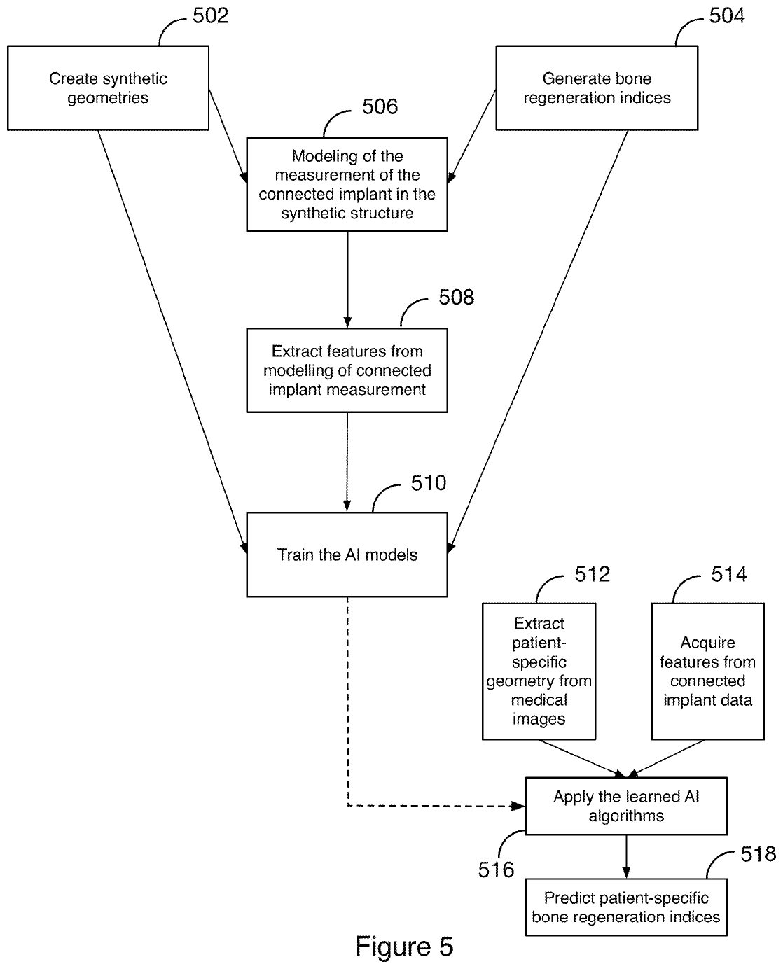

FIG. 5 is a flow chart illustrating an example embodiment of a method for providing assessment and analysis of a medical patient, in accordance with some aspects of the systems and methods herein. The example embodiment shows an offline process for training AI models. Steps 502, 504, 506, 508, and 510 constitute the training phase for training AI models, whereas acts 512, 514, 516, and 518 constitute the prediction phase.

In steps 502 and 512, patient-specific geometry is extracted or created from data. For example, the geometry may be vertebral and disc, bone ends and callus, maxillofacial bone or other bone geometries. In some embodiments, for generating synthetic geometries in step 502, data could be in the form of altering existing models. Alternatively, data could be created without any extraction from medical images. Existing models could be created from one or more patients' medical images to obtain a large number of models. In some embodiments, the number of synthetic models could amount to several hundred of thousand models. In other embodiments, however, the dataset could amount to fewer or larger synthetic models' quantities. The alteration of the models could be, e.g., integration, removal or modification of dimensions, defect, hole, micro-cracks, cracks, porosity or other alterations. These geometrical properties could be randomly or systematically altered. In some embodiments, these geometrical properties could be used to define, e.g., one or more bone regeneration indices in step 504. In some embodiments, for generating patient specific geometries in step 512, data could be extracted from medical images. In some embodiments, one or more AI models could be used to extract the patient specific geometries from medical images.

In step 504, bone regeneration indices are generated. Bone regeneration indices could be from two types. Firstly, some bone regeneration indices represent physical or chemical properties which could be mechanical properties, or alternatively, e.g., dielectrics, thermics, electrostatics, magnetostatics properties or potential of hydrogen. Mechanical properties of the one or more different tissues represented by the geometry generated in act 502 could be a combination of, e.g., elastic, viscoelastic, hyperelastic, poroelastic, elastoplastic or other mechanical behavior. Mechanical properties could be local or global or both local and global properties. Mechanical behavior could describe the behavior of the tissue of interest subjected to a loading in, e.g., tensile, compression, bending, torsion, vibration or other loadings. Secondly, some bone regeneration indices represent bone defect assessment, and are computed as linear or more complex functions of the amount, the shape, the space repartition of defect, hole, micro-cracks, cracks or other abnormal geometries while considering geometrical dimension, density and the porosity of the models.

In step 508, measures of interest are computed from a connected implant measurement modeling (step 506) based on synthetic geometry obtained in step 502, physical and chemical properties obtained in step 504, as well as exterior solicitations comparable to those used to measure features of interest using the connected implant in step 514. Exterior solicitations could be, e.g., impact, force, displacement, electromagnetic signal or any other source of exterior solicitations. In the preferred embodiment, the biomechanical model could be a finite element method model. Alternatively, it could be a gradient discretization method, finite difference method, discrete element method, meshfree methods, computational fluid dynamics or any other numerical method for computing a biomechanical model or any other suitable physical and/or chemical model.

In step 510, one or more AI models are trained taking into input synthetic geometry obtained in step 502, measures of interest obtained in step 508 and outputting (or taking as ground truth) bone regeneration indices (step 504). In another embodiment, external solicitation previously described could also be set as an input for AI. As described above, AI models could be several multilayer neural networks or any other AI algorithms.

In step 514, measures of interest are acquired from a connected implant in a new patient. In some embodiments, measures of interest could be raw signals in the frequency or time domain. Alternatively, measures of interest could be precomputed values such as force, stress, displacement, or any other values or indices at one or more locations.

In step 516, AI models which were trained in step 510 are applied on patient specific geometries acquired in step 512, and measures of interest are acquired from the connected implant in step 514, outputting bone regeneration indices prediction in step 518.

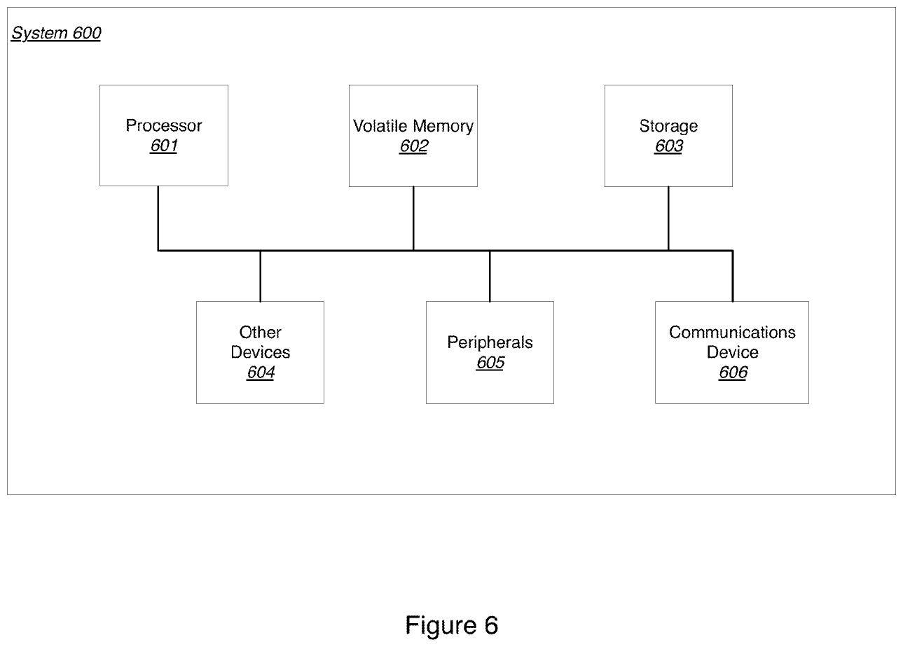

FIG. 6 is a diagram illustrating an exemplary computer that may perform processing in some embodiments. Exemplary computer 600 may perform operations consistent with some embodiments. The architecture of computer 600 is exemplary. Computers can be implemented in a variety of other ways. A wide variety of computers can be used in accordance with the embodiments herein. In some embodiments, cloud computing components and/or processes may be substituted for any number of components or processes illustrated in the example.

Processor 601 may perform computing functions such as running computer programs. The volatile memory 602 may provide temporary storage of data for the processor 601. RAM is one kind of volatile memory. Volatile memory typically requires power to maintain its stored information. Storage 603 provides computer storage for data, instructions, and/or arbitrary information. Non-volatile memory, which can preserve data even when not powered and including disks and flash memory, is an example of storage. Storage 603 may be organized as a file system, database, or in other ways. Data, instructions, and information may be loaded from storage 603 into volatile memory 602 for processing by the processor 601.

The computer 600 may include peripherals 605. Peripherals 605 may include input peripherals such as a keyboard, mouse, trackball, video camera, microphone, and other input devices. Peripherals 605 may also include output devices such as a display. Peripherals 605 may include removable media devices such as CD-R and DVD-R recorders/players. Communications device 606 may connect the computer 100 to an external medium. For example, communications device 606 may take the form of a network adapter that provides communications to a network. A computer 600 may also include a variety of other devices 604. The various components of the computer 600 may be connected by a connection medium 610 such as a bus, crossbar, or network.

While the invention has been particularly shown and described with reference to specific embodiments thereof, it should be understood that changes in the form and details of the disclosed embodiments may be made without departing from the scope of the invention. Although various advantages, aspects, and objects of the present invention have been discussed herein with reference to various embodiments, it will be understood that the scope of the invention should not be limited by reference to such advantages, aspects, and objects. Rather, the scope of the invention should be determined with reference to patent claims.

What is claimed:

1. A method for providing assessment and analysis of a medical patient, comprising:
receiving medical imaging data associated with the patient;
receiving connected implant data from an implant device implanted in the patient, the implant device comprising one or more sensors;
extracting, via one or more artificial intelligence (AI) models, one or more features of interest from the medical imaging data and connected implant data; and
generating one or more reports based on the extracted features of interest.

2. The method of claim 1, further comprising:
determining one or more matching similarities, wherein the determining comprises comparing the one or more extracted features of interest to one or more other features of interest from previous patient data associated with one or more additional patients,
wherein the generating of the one or more reports is further based on the one or more matching similarities.

3. The method of claim 1, further comprising:
receiving invasive patient data associated with the patient,
wherein the one or more features of interest are further extracted from the invasive patient data.

4. The method of claim 1, further comprising:
receiving non-invasive patient data associated with the patient,
wherein the one or more features of interest are further extracted from the non-invasive patient data.

5. The method of claim 1, further comprising:
generating a set of medical prediction indices based on the plurality of extracted features,
wherein the one or more reports comprise at least a subset of the medical prediction indices.

6. The method of claim 1, further comprising:
training the one or more artificial intelligence (AI) models to perform one or more tasks, wherein the one or more tasks comprise at least extracting the one or more features of interest.

7. The method of claim 6, wherein training the one or more AI models is performed using one or more transfer learning methods, wherein each transfer learning method has its own transfer learning dataset, and wherein the one or more transfer learning datasets are unrelated to the one or more tasks.

8. The method of claim 6, wherein training the one or more AI models is performed using one or more datasets based on synthetic data, and wherein the synthetic data is related to one or more synthetic models.

9. The method of claim 8, wherein training the one or more AI models further comprises:
generating patient-specific synthetic geometries based on features extracted from the medical imaging data;
generating one or more indices comprising physical or chemical properties of the generated synthetic geometries;
generating one or more synthetic models based on the synthetic geometries and the indices;
extracting one or more measures from the one or more synthetic models, wherein the measures are similar or identical to those used to measure features of interest using the connected implant; and
training the algorithm to output indices from the synthetic geometries and the measures.

10. The method of claim 9, wherein the one or more indices are bone regeneration indices.

11. The method of claim 1, further comprising:
storing the one or more reports in one or more patient-specific medical records.

12. The method of claim 1, wherein the one or more features of interest relate to bone regeneration, and wherein the one or more reports comprise a plurality of bone regeneration metrics.

13. The method of claim 12, further comprising:
initializing one or more distraction osteogenesis parameters;
predicting one or more bone regeneration indices based on the distraction osteogenesis parameters and the one or more bone regeneration metrics; and
generating optimized distraction osteogenesis parameters based on the predicted bone regeneration indices and the one or more bone regeneration metrics.

14. A non-transitory computer-readable medium containing instructions for providing assessment and analysis of a medical patient, comprising:
instructions for receiving medical imaging data associated with the patient;
instructions for receiving connected implant data from an implant device implanted in the patient, the implant device comprising one or more sensors;
instructions for extracting, via one or more artificial intelligence (AI) models, one or more features of interest from the medical imaging data and connected implant data; and
instructions for generating one or more reports based on the extracted plurality of features.

15. The non-transitory computer-readable medium of claim 14, further comprising:
instructions for determining one or more matching similarities, wherein the determining comprises comparing the extracted one or more features of interest to one or more other features of interest from previous patient data associated with one or more additional patients,
wherein the generating of the one or more reports is further based on the plurality of matching similarities.

16. The non-transitory computer-readable medium of claim 14, further comprising:
instructions for receiving invasive patient data associated with the patient,
wherein one or more features of interest are further extracted from the invasive patient data.

17. The non-transitory computer-readable medium of claim 14, further comprising:
instructions for receiving non-invasive patient data associated with the patient,
wherein one or more features of interest are further extracted from the non-invasive patient data.

18. The non-transitory computer-readable medium of claim 14, further comprising:
instructions for extracting, based on one or more matching similarities, one or more similar images, wherein the similar images have similar features to at least a subset of the one or more medical images of the patient,
wherein the generated report comprises the one or more similar images.

19. The non-transitory computer-readable medium of claim 14, further comprising:
instructions for generating a set of medical prediction indices based on one or more matching similarities,
wherein the one or more reports comprise at least a subset of the medical prediction indices.

20. The non-transitory computer-readable medium of claim 14, further comprising:
instructions for training the one or more artificial intelligence (AI) models to perform one or more tasks, wherein the one or more tasks comprise at least extracting one or more features of interest.

21. The non-transitory computer-readable medium of claim 14, wherein the plurality of features relate to bone regeneration, wherein the one or more reports comprise a plurality of bone regeneration metrics

22. The method of claim 21, further comprising:
instructions for initializing one or more distraction osteogenesis parameters;
instructions for predicting one or more bone regeneration indices based on the distraction osteogenesis parameters and one or more bone regeneration metrics; and
instructions for generating optimized distraction osteogenesis parameters based on the predicted bone regeneration indices and the one or more bone regeneration metrics.
